(19) United States
(12) Patent Application Publication
(10) Pub. No.: US 2004/0137468 A1
(43) Pub. Date: Jul. 15, 2004

(54) Solid phase based nucleic acid assays combining high affinity and high specificity

(76) Inventors: Brian Warner, Martinez, CA (US); John Quinn, Concord, CA (US); Jens Burmeister, Koln (DE); Ingmar Dorn, Koln (DE); Edgar Diessel, Koln (DE)

Correspondence Address: COZEN O'CONNOR, P.C., 1900 MARKET STREET, PHILADELPHIA, PA, 19103-3508, US

(21) Appl. No.: 10/651,416

(22) Filed: Aug. 29, 2003

Related U.S. Application Data
(60) Provisional application No. 60/407,468, filed on Aug. 30, 2002.

(51) Int. Cl. C12Q 1/68; C12M 1/34

(52) U.S. Cl. 435/6; 435/287.2

(57) ABSTRACT

The invention relates to methods for detection of nucleic acids on a solid phase, combining high affinity and high specificity. More particularly, the invention relates to methods combining high-affinity hybridization with highly specific enzymatic discrimination in solid phase based nucleic acid assays. This invention further relates to kits containing the reagents necessary for carrying out the disclosed assays.

### CROSS REFERENCE TO RELATED APPLICATIONS

This application claims priority to U.S. Provisional Application No. 60/407,468, filed Aug. 30, 2002, which is incorporated herein by reference.

### TECHNICAL FIELD

This invention relates to methods for detection of nucleic acids on a solid phase with high affinity and high specificity. More particularly, the invention relates to methods combining high-affinity hybridization with highly specific enzymatic discrimination in solid phase based nucleic acid assays. This invention further relates to kits containing the reagents necessary for carrying out the disclosed assays. The detection of deoxyribonucleic acid (DNA) or ribonucleic acid (RNA) is of importance in human or veterinary diagnostics, food control, environmental analysis, crop protection, biochemical/pharmacological research, or forensic medicine.

### BACKGROUND OF THE INVENTION

In a typical solid phase based nucleic acid assay, capture oligonucleotides are immobilized on a solid support. The labeled or unlabeled nucleic acid target is specifically hybridized to the capture probes. After hybridization and, if necessary, labeling, the hybridization event can be detected using e.g. optical, electrical, mechanical, magnetic or other readout methods. Generally, the high specificity of base pairing interactions between strands of nucleic acids are used in these methods to differentiate between different targets. Using a solid phase enables facile multiplexing of nucleic acid hybridization assays by spatially separating different capture oligonucleotides having different sequences. In addition, the solid phase facilitates separation of bound and unbound species by simple washing steps. A huge number of different supports e.g. planar surfaces (“chips”), beads or gel matrices can be used as solid phases. Methods for preparation of DNA oligonucleotide arrays are summarized e.g. in S.L. Beaucage, Curr. Med. Chem. 2001, 8, 1213-1244 or M. C. Pirrung, Angew. Chem. 2002, 114, 1326-1341. Solid phase based nucleic acid hybridization assays are widely used e.g. for analysis of single nucleotide polymorphisms (SNPs), expression profiling or viral detection (for a summary see e.g. J. Wang, Nucl. Acids Res. 2000, 28, 3011-3016).

An alternative approach for specific detection of nucleic acids employs the specificity of enzymes for the discrimination of different probe-target complexes on solid phases or in solution. For example, in enzymatic SNP assays, the immobilized capture probe can be used as primer for allele-specific primer extension reactions or as one component of allele-specific oligonucleotide ligation reactions. Ligation assays are described e.g. in U.S. Pat. No. 5,800,994 and WO 9631622; primer extension reactions are described e.g. in WO A200058516 /U.S. Pat. No. 200,104,6673 /EP 1061135A2. Again, multiplexing of enzymatic nucleic acid assays can be achieved by spatial separation of oligonucleotide probes on a surface.

Chemical oligonucleotide ligation reactions can be used for discrimination between different sequences, analogous to the enzymatic methods mentioned above. For example, WO 9424143 describes chemical ligation of an (α-haloacetyl derivatized oligonucleotide to a second phosphorothioate modified oligonucleotide, spontaneously and selectively forming a covalent bond.

Methods for genotyping single nucleotide polymorphisms are described e.g. in P.-Y. Kwok, Annu. Rev. Genomics Hum. Gen. 2001, 2, 235-258. One current general method for detection of SNPs relies on a three step procedure: purification of genomic DNA from biological material, amplification of the desired gene fragment e.g. by PCR and subsequent detection e.g. by allele specific hybridization, enzymatic reactions etc. Due to the current lack of highly sensitive nucleic acid detection methods, the amplification step is unavoidable. However, this step is very laborious, time consuming, expensive and difficult to multiplex. Therefore, there is a need for assays that allow for highly sensitive, highly selective detection of nucleic acids, e.g. containing SNPs, directly from genomic DNA, without prior amplification.

The present invention is directed to a method for combining high specificity with high sensitivity in order to enable nucleic acid analysis on a solid surface from biological sources without prior amplification.

A solid phase based nucleic acid detection method that employs electrical current to control hybridization reactions is disclosed in WO 9512808. Using electrical current, nucleic acids are actively transported from solution to specific locations on a surface, addressed by electrodes. The method can be used to control and enhance the specificity and sensitivity of nucleic acid hybridization reactions. One serious drawback of this technology is electrolysis that accompanies the electronic addressing process. Thus, a restriction to certain buffer systems exists that imposes the necessity of sample preparation steps. In addition, each hybridization event has to be addressed individually. Therefore, the complexity of electrode structures on the surface increases with the number of analytes to be detected.

An example of a nucleic acid assay which employs multiple hybridization reactions for combination of affinity and specificity is given in WO 95/16055. In this approach, capture probes are bound to a surface. One or more capture extender molecules are employed, each containing a target specific binding sequence and a support binding sequence able to hybridize to the surface bound capture probes. The capture extender sequences are used to bind the target to the support with high affinity. For detection, e.g. amplification multimers are hybridized to the target in order to amplify signals. Different sequences can be discriminated by specific hybridization of capture extenders containing sequences specific to different target regions. In case of targets that differ in their sequences by only one base (e.g. SNPs) this approach does not work for more than one capture extender, because the differences in thermodynamic stabilities and thus melting temperatures are too small for effective discrimination.

A nucleic acid hybridization assay combining affinity and specificity is described by Wanda L.B. White et al. (Poster: “SNP determination by dual hybridization with DNA and PNA probes”, Cambridge Healthtech Institute Conference on Nucleic Acid Based Technologies, Washington D.C., 2002). An immobilized 40 mer DNA is used to capture the target with high affinity, while a short PNA probe is used for allele-specific hybridization. Drawbacks of this assay principle are (1) long capture sequences are not specific enough for multiplexed assays and (2) for analysis of SNPs a second multiplexing principle (e.g. different colored labels) besides the solid support has to be introduced in order to differentiate between alleles.

In summary, many nucleic acid assay formats, that make use of a hybridization reaction of a target probe to a capture probe immobilized on a solid phase, suffer from either sensitivity or selectivity. Therefore, problems occur if e.g. single nucleotide polymorphisms must be detected in samples without prior target amplification. If the capture probes are designed for maximum affinity and therefore sensitivity of the assay the capturing reaction suffers from selectivity. If the capture probes are designed for maximum selectivity the hybridizaton reaction displays only moderate affinity.

### SUMMARY OF THE INVENTION

Methods and kits are provided for detecting nucleic acids with high sensitivity and high specificity on a solid support. In general, the methods combine high affinity capture using one or more target specific oligonucleotides with highly specific enzymatic discrimination methods. Preferred methods include the use of one or more capture extender molecules for capturing the target with high affinity, in combination with a “discrimination extender” that is used for enzymatic reactions like ligations or primer extensions thereby specifically incorporating a label.

### BRIEF DESCRIPTION OF THE DRAWINGS

FIG. 1. A summary of the assay in a preferred embodiment. 3′-terminally blocked capture extenders are capturing the target, unblocked discrimination extenders are used for specific discrimination. All capture probes are immobilized via their 3′-termini.

FIG. 2. A summary of the assay in another preferred embodiment, using enzymatic ligation for discrimination. Capture extenders are capturing the target, 5′-phosphorylated discrimination extenders are used for specific discrimination. All capture probes are immobilized via their 5′-termini.

FIG. 3. A summary of the assay in another preferred embodiment. All 3′ termini except the 3′-terminus that is used for enzymatic discrimination (discrimination extender) are either blocked or immobilized.

FIG. 4. A summary of the assay in another preferred embodiment. 3′ terminally blocked capture probes are used for capturing the target. An additional unblocked discrimination probe is used for enzymatic discrimination.

FIG. 5. A summary of the assay in another preferred embodiment. 5′-terminally immobilized capture probes are used for capturing the target. A 5′-phosphorylated discrimination probe is used for enzymatic discrimination.

FIG. 6. One example for multiplexing of the assay on a planar surface. Capture probes are immobilized in separate spots. Allele-specific discrimination extenders are hybridized to the respective spots prior to hybridization of the target.

FIG. 7. Another example for multiplexing of the assay on a planar surface. Capture probes are immobilized in separate spots. Capture extenders and allele-specific discrimination extenders are hybridized to the respective spots.

### DETAILED DESCRIPTION OF THE INVENTION

The invention combines high-affinity oligonucleotide capture with highly specific enzymatic discrimination on a solid support, preferably for the detection of single nucleotide polymorphisms in multiplex assays without prior amplification of genomic DNA. The invention makes use of the fact that enzymatic reactions like polymerase mediated primer extension or ligase mediated oligonucleotide ligation proceed via nucleophilic attack of the free 3′-terminal hydroxyl group on activated 5 5′-terminal phosphate groups of a nucleotide or oligonucleotide, thereby forming a 3′-5′-phosphodiester bond. Therefore, 3′-terminal hydroxyl groups can be easily prevented from polymerase or ligase extensions by blocking. In the disclosed assay format, all oligonucleotides, except for the discrimination extender that is used for enzymatic discrimination, are blocked on their 3′-termini. Capture probes can be blocked against enzymatic reactions by immobilization via their 3′-hydroxyl-termini, eventually employing spacer groups between the 3′-terminus and the group used for immobilization. Other 3′-termini can be blocked against enzymatic processing by using e.g. 3′-deoxynucleotides, 2′,3′-dideoxynucleotides, 3′-phosphates, 3′-aminoalkylphosphates, 3′-alkylphosphates, 3′-carboxyalkylphosphates, 3′-terminal biotin modifications, 3′-terminal inverted nucleotides etc. All modifications mentioned above and other possible blocking modifications can be incorporated using standard oligonucleotide synthesis methods.

The discrimination reaction employed in the disclosed assays can be an enzymatically catalyzed primer extension or oligonucleotide ligation reaction. Alternatively, nonenzymatic, chemical extension methods can be used to achieve allele-specific incorporation of labeling entities. The fidelity of some chemical reactions for oligonucleotide ligation is comparable to enzymatic methods, for an example see K.D. James, A. D. Ellington, Chem. Biol. 1997, 4, 595-605. Enzymatic discrimination relies on a primer that is the perfect complement of one allele sequence. The position of the SNP is preferably situated at the 3′-terminal nucleotide of the primer. In case of ligation reactions the discrimination extender can display an unblocked 3′-terminus with the 3′-terminal nucleotide being complementary to the SNP position. A second, 5′-phosphorylated, labeled oligonucleotide, being complementary to a region of the target neighboring the SNP, is ligated in a ligase mediated reaction thereby introducing the labeling entity. Alternatively, the discrimination extender used for ligase mediated discrimination can display a phosphorylated 5′-terminus with the 5′-terminal nucleotide being complementary to the SNP position. A second, 3′-terminally unblocked labeled oligonucleotide, being complementary to a region of the target neighboring the SNP, is ligated in a ligase mediated reaction thereby introducing the labeling entity.

Using the enzymatic discrimination reaction a number of different labeling entities or entities that allow for labeling reactions, can be specifically incorporated. Since multiplexing of the assay is achieved by spatial separation of discrimination extenders, only one type of label is necessary for the disclosed assays, if SNP analysis is being performed. Labels or groups enabling labeling reactions can be e.g. fluorophors, nanoparticles, redox active moieties, antibodies, antibody fragments, biotin, aptamers, peptides, proteins, mono- or polysaccharides, nucleic acids, nucleic acid analogs, complexing agents, cyclodextrins, crown ethers, anticalins, receptors etc.

Depending on the type of label that has been introduced during the enzymatic or chemical reaction, different readout methods can be used to assess the result of the assay. Examples for readout methods include optical, electrical, mechanical or magnetic detection. More specifically, fluorophores can be detected using e.g. planar optical waveguides as disclosed in U.S. Pat. No. 5,959,292 and WO 99/47705, total reflection on interfaces as disclosed in DE 196 28 002 or using optical fibers as disclosed in U.S. Pat. No. 4,815,843. Nanoparticle labels can be detected e.g. via optical methods or e.g. by direct electrical detection after autometallographic enhancement as disclosed in U.S. patents U.S. Pat. No. 4,794,089, U.S. Pat. No. 5,137,827 and U.S. Pat. No. 5,284,748.

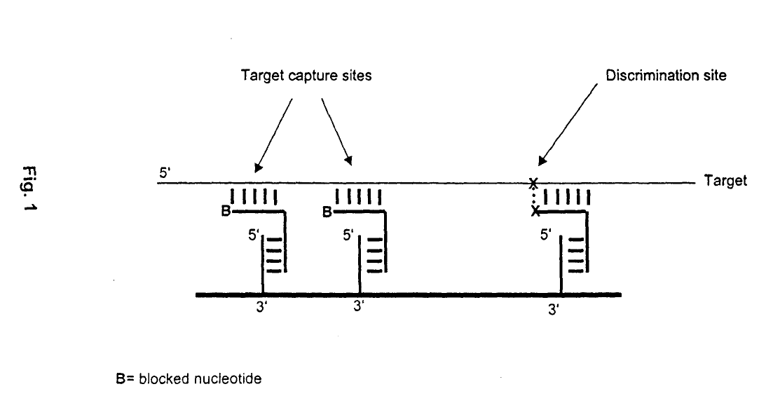

In a first aspect of the invention, an assay is provided in which one or more capture extender molecules are used, each of which must bind to the target molecule at a specific site (FIG. 1). The 3′-termini of these capture extenders are blocked in order to prevent enzymatic extension or ligation. Additional discrimination extenders are used, each of which is complementary to one allele of the target. The SNP is positioned at the 3′-terminal nucleotide of these discrimination extenders that are used for enzymatic discrimination. All capture probes are immobilized on the solid support via their 3′-termini. In order to achieve spatial addressing, the discrimination extenders that are used for allelic discrimination have to be hybridized to the support prior to hybridization of the target. The capture extenders can be mixed with the target in solution prior to hybridization. Alternatively, all capture extenders can be hybridized to the immobilized capture probes prior to hybridization of the target.

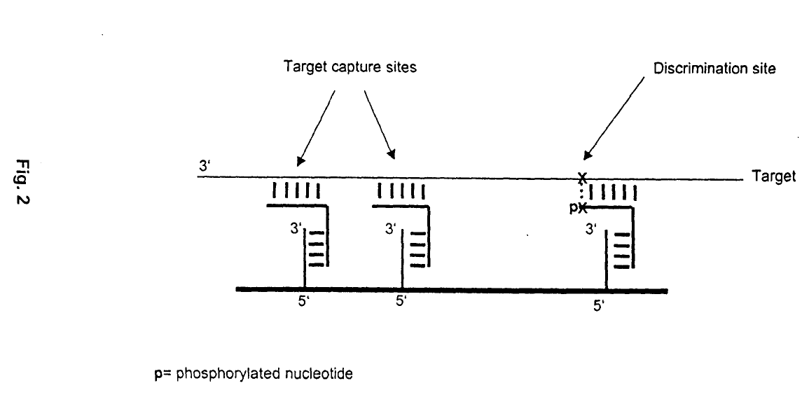

In a second aspect of the invention, an assay is provided in which one or more capture extender molecules are used, each of which must bind to the target molecule at a specific site (FIG. 2). Additional discrimination extenders are used, each of which is complementary to one allele of the target and carries a 5′-terminal phosphorylated hydroxyl group. The SNP is positioned at the 5′-terminal nucleotide of these discrimination extenders that are used for enzymatic discrimination. All capture probes are immobilized on the solid support via their 5′-termini. In order to achieve spatial addressing, the discrimination extenders that are used for allelic discrimination have to be hybridized to the support prior to hybridization of the target. The capture extenders can be mixed with the target in solution prior to hybridization. Alternatively, all capture extenders can be hybridized to the immobilized capture probes prior to hybridization of the target.

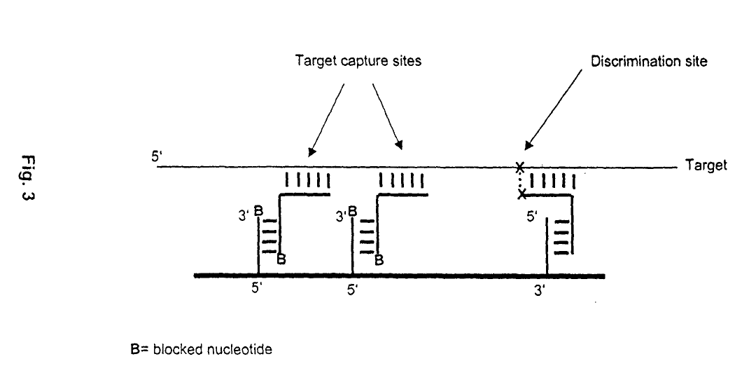

In a third aspect of the invention, an assay is provided in which one or more capture extender molecules are used, each of which must bind to the target molecule at a specific site (FIG. 3). The 3′-termini of these capture extenders, as well as the 3′-termini of the immobilized capture probes, are blocked in order to prevent enzymatic extension or ligation. Additional discrimination extenders are used, each of which is complementary to one allele of the target. The SNP is positioned at the 3′-terminal nucleotide of these discrimination extenders that are used for enzymatic discrimination. Those capture probes, that are complementary to the capture extenders, are immobilized on the solid support via their 5′-termini. The capture probes complementary to the discrimination extenders are immobilized to the solid support via their 3′-termini. In order to achieve spatial addressing, the discrimination extenders that are used for allelic discrimination have to be hybridized to the support prior to hybridization of the target. The capture extenders can be mixed with the target in solution prior to hybridization. Alternatively, all capture extenders can be hybridized to the immobilized capture probes prior to hybridization of the target.

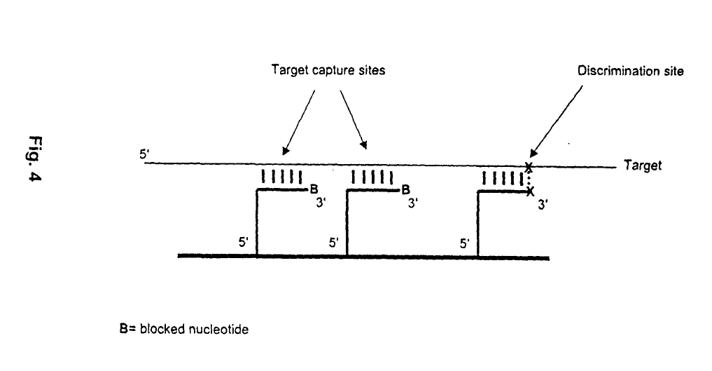

In a fourth aspect of the invention, an assay is provided in which one or more capture probe molecules are used, each of which must bind to the target molecule at a specific site (FIG. 4). The capture probes are immobilized via their 5′-termini, their 3′-ends are blocked to prevent enzymatic extension or ligation. In addition, discrimination probes are bound to the surface, each of which is complementary to one allele of the target.

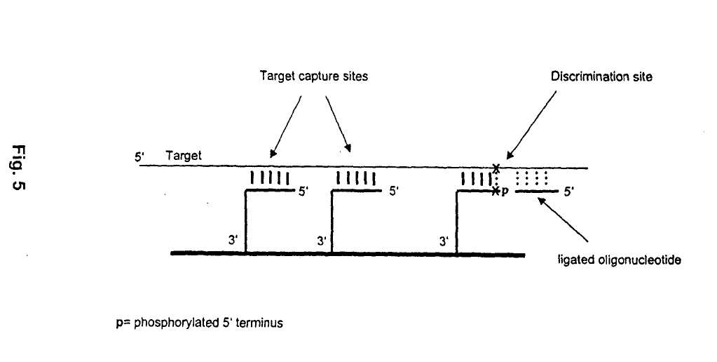

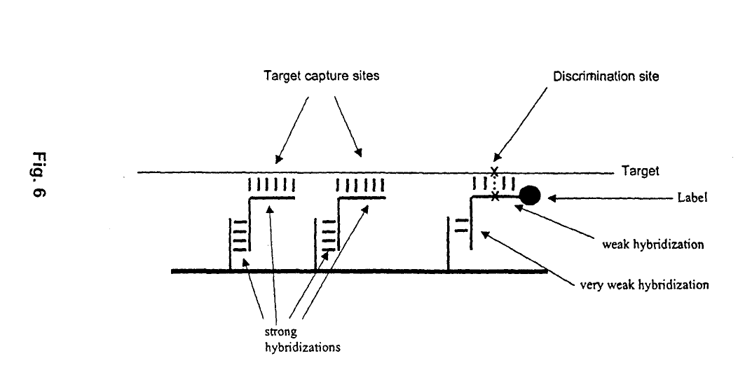

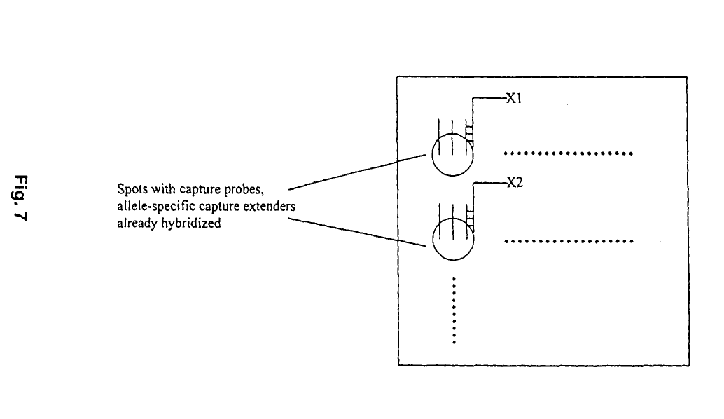

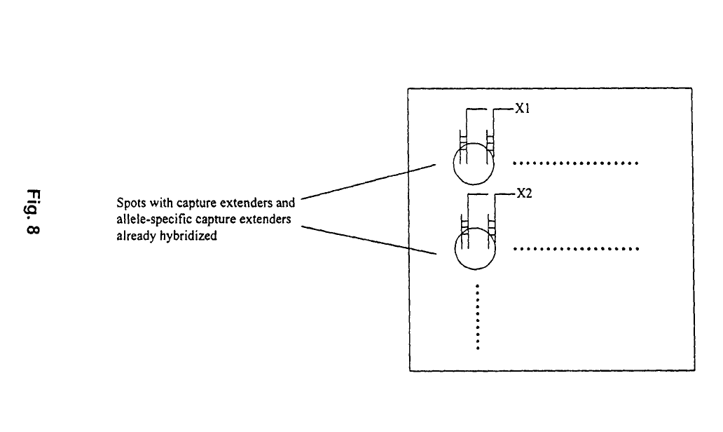

In a fifth aspect of the invention, an assay is provided in which one or more capture probe molecules are used, each of which must bind to the target molecule at specific site (FIG. 5). The capture probes are immobilized via their 3′-termini. In addition, discrimination probes are bound to the surface, each of which is complementary to one allele of the target. These allele specific discrimination probes bear phosphorylated 5′-termini, allowing for enzymatic ligation of labeled oligonucleotides.

All references, patents and published patent applications referred to herein are hereby incorporated by reference.

We claim:

1. A solid phase based nucleic acid assay that can distinguish alleles that comprise a single nucleotide polymorphism in a sample containing a target nucleic acid molecule comprising
a) immobilizing the target nucleic acid molecule to a solid support using
i) one or more capture oligonucleotides that are immobilized to the solid support, that hybridize to the target molecule and that have terminal nucleotides blocked and/or unphosphorylated terminal nucleotides and
ii) a discrimination oligonucleotide that are immobilized to the solid support, that hybridize to the target molecule wherein a nucleotide at a terminus of the discrimination oligonucleotide is complementary to the single nucleotide polymorphism position and is unblocked and/or phosphorylated;
b) performing a reaction on any unblocked and/or phosphorylated terminal nucleotide hybridized to a complementary nucleotide, the reaction being specific for unblocked and/or phosphorylated terminal nucleotide perfectly hybridized to a complementary nucleotide; and
c) determining if any reaction occurred, wherein detecting a reaction of unblocked and/or phosphorylated terminal nucleotide perfectly hybridized to a complementary nucleotide indicates the discrimination oligonucleotide hybridized perfectly to the target nucleic acid molecule at the single nucleotide polymorphism position.

2. A solid phase based nucleic acid assay of claim 1 comprising:
a) contacting under hybridizing conditions a target nucleotide molecule, one or more target capture extenders, a discrimination extender and a solid support that comprises one or more target capture probes and a capture probe immobilized at the 3′ terminus to the solid support directly or with spacers, wherein:
i) the discrimination extender comprises a sequence that is complementary to a sequence of the capture probe sufficient to immobilize the discrimination extender to the solid support, and a sequence that is complementary to a sequence of the target nucleic acid molecule including a sequence complementary to an allele that comprises a single nucleotide polymorphism in which the nucleotide complementary to the single nucleotide polymorphism position is at an unblocked 3′ terminal nucleotide of the discrimination extender,
ii) the capture probe comprises a sequence that is complementary to a sequence of the discrimination extender sufficient to immobilize the discrimination extender to the solid support,
iii) the one or more target capture probes comprise a sequence that is complementary to a sequence of one or more target capture extenders each sufficient to immobilize the target capture extender to the solid support, each target capture extender further comprising a sequence that is complementary to a sequence of the target nucleic acid molecule each sufficient to hybridize the target nucleic acid molecule to the target capture extender, each target capture extender having a blocked 3′ terminal nucleotide; and
iv) the one or more target capture extenders hybridize to the one or more target capture probes on the solid support and to the target nucleic acid molecule, and the discrimination extender hybridizes to the capture probe on the solid support and to the target nucleic acid molecule;
b) performing a reaction on any unblocked 3′ terminal nucleotide hybridized to a complementary nucleotide, the reaction being specific for unblocked 3′ terminal nucleotide perfectly hybridized to a complementary nucleotide; and
c) determining if any reaction occurred, wherein detecting a reaction of unblocked 3′ terminal nucleotide perfectly hybridized to a complementary nucleotide indicates the discrimination extender hybridized perfectly to the target nucleic acid molecule at the 3′ terminal nucleotide and that the allele present in the target nucleic acid molecule is the allele that has the single nucleotide polymorphism complementary to the 3′ terminus of the discrimination extender.

3. A solid phase based nucleic acid assay of claim 1 comprising:
a) contacting under hybridizing conditions a target nucleotide molecule, one or more target capture extenders, a discrimination extender and a solid support that comprises one or more target capture probes and a capture probe immobilized at the 5′ terminus to the solid support directly or with spacers, wherein:
i) the discrimination extender comprises a sequence that is complementary to a sequence of the capture probe sufficient to immobilize the discrimination extender to the solid support, and a sequence that is complementary to a sequence of the target nucleic acid molecule including a sequence complementary to an allele that comprises a single nucleotide polymorphism in which the nucleotide complementary to the single nucleotide polymorphism position is at a phosphorylated 5′ terminal nucleotide of the discrimination extender,
ii) the capture probe comprises a sequence that is complementary to a sequence of the discrimination extender sufficient to immobilize the discrimination extender to the solid support,
iii) the one or more target capture probes comprise a sequence that is complementary to a sequence of one or more target capture extenders each sufficient to immobilize the target capture extender to the solid support, each target capture extender further comprising a sequence that is complementary to a sequence of the target nucleic acid molecule each sufficient to hybridize the target nucleic acid molecule to the target capture extender, each target capture extender having an unphosphorylated 5′ terminal nucleotide; and
iv) the one or more target capture extenders hybridize to the one or more target capture probes on the solid support and to the target nucleic acid molecule, and the discrimination extender hybridizes to the capture probe one the solid support and to the target nucleic acid molecule;
b) performing a reaction on any phosphorylated 5′ terminal nucleotide hybridized to a complementary nucleotide, the reaction being specific for phosphorylated 5′ terminal nucleotide perfectly hybridized to a complementary nucleotide; and
c) determining if any reaction occurred, wherein detecting a reaction of unblocked 3′ terminal nucleotide perfectly hybridized to a complementary nucleotide indicates the discrimination extender hybridized perfectly to the target nucleic acid molecule at the phosphorylated 5′ nucleotide and that the allele present in the target nucleic acid molecule is the allele that has the single nucleotide polymorphism complementary to the 5′ terminus of the discrimination extender.

4. A solid phase based nucleic acid assay of claim 1 comprising:
a) contacting under hybridizing conditions a target nucleotide molecule, one or more target capture extenders, a discrimination extender, and a solid support that comprises one or more target capture probes and a capture probe immobilized at the 3′ terminus to the solid support directly or with spacers and one or more target capture probes immobilized at the 5′ terminus to the solid support directly or with spacers, wherein:
i) the discrimination extender comprises a sequence that is complementary to a sequence of the capture probe sufficient to immobilize the discrimination extender to the solid support, and a sequence that is complementary to a sequence of the target nucleic acid molecule including a sequence complementary to an allele that comprises a single nucleotide polymorphism in which the nucleotide complementary to the single nucleotide polymorphism position is at an unblocked 3′ terminal nucleotide of the discrimination extender,
ii) the capture probe comprises a sequence that is complementary to a sequence of the discrimination extender sufficient to immobilize the discrimination extender to the solid support,
iii) one or more target capture probes comprise a sequence that is complementary to a sequence of one or more target capture extenders each sufficient to immobilize the target capture extender to the solid support, each target capture extender further comprising a sequence that is complementary to a sequence of the target nucleic acid molecule each sufficient to hybridize the target nucleic acid molecule to the target capture extender, each target capture extender and each target capture probe having a blocked 3′ terminal nucleotide; and
iv) the one or more target capture extenders hybridize to the one or more target capture probes on the solid support and to the target nucleic acid molecule, and the discrimination extender hybridizes to the capture probe on the solid support and to the target nucleic acid molecule;
c) performing a reaction on any unblocked 3′ terminal nucleotide hybridized to a complementary nucleotide, the reaction being specific for unblocked 3′ terminal nucleotide perfectly hybridized to a complementary nucleotide; and
d) determining if any reaction occurred, wherein detecting a reaction of unblocked 3′ terminal nucleotide perfectly hybridized to a complementary nucleotide indicates the discrimination extender hybridized perfectly to the target nucleic acid molecule at the 3′ terminal nucleotide and that the allele present in the target nucleic acid molecule is the allele that has the single nucleotide polymorphism complementary to the 3′ terminus of the discrimination extender.

5. A solid phase based nucleic acid assay of claim 1 comprising:
a) contacting under hybridizing conditions a target nucleic acid molecule and a solid support that comprises one or more target capture probes and a discrimination probe linked to the solid support at the 5′ terminus directly or with spacers, wherein:
i) the discrimination probe comprises a sequence that is complementary to a sequence of the target nucleic acid molecule including a sequence complementary to an allele that comprises a single nucleotide polymorphism in which the nucleotide complementary to the single nucleotide polymorphism position is at an unblocked 3′ terminal nucleotide of the discrimination probe,
ii) the one or more target capture probes comprise a sequence that is complementary to one or more sequences of the target nucleic acid molecule each sufficient to hybridize the target nucleic acid molecule to the capture probe, each capture probe having a blocked 3′ terminal nucleotide;
b) performing a reaction on any unblocked 3′ terminal nucleotide hybridized to a complementary nucleotide, the reaction being specific for unblocked 3′ terminal nucleotide perfectly hybridized to a complementary nucleotide; and
d) determining if any reaction occurred, wherein detecting a reaction of unblocked 3′ terminal nucleotide perfectly hybridized to a complementary nucleotide indicates the discrimination probe hybridized perfectly to the target nucleic acid molecule at the 3′ terminal nucleotide and that the allele present in the target nucleic acid molecule is the allele that has the single nucleotide polymorphism complementary to the 3′ terminus of the discrimination probe.

6. A solid phase based nucleic acid assay of claim 1 comprising:
a) contacting under hybridizing conditions a target nucleic acid molecule and a solid support that comprises one or more target capture probes and a discrimination probe linked to the solid support at the 3′ terminus directly or with spacers, wherein:
i) the discrimination probe comprises a sequence that is complementary to a sequence of the target nucleic acid molecule including a sequence complementary to an allele that comprises a single nucleotide polymorphism in which the nucleotide complementary to the single nucleotide polymorphism position is at a phosphorylated 5′ terminal nucleotide of the discrimination probe,
ii) the one or more target capture probes comprise a sequence that is complementary to one or more sequences of the target nucleic acid molecule each sufficient to hybridize the target nucleic acid molecule to the capture probe, each capture probe having an unphosphorylated 5′ terminal nucleotide;
b) performing a reaction on any phosphorylated 5′ terminal nucleotide hybridized to a complementary nucleotide, the reaction being specific for phosphorylated 5′ terminal nucleotide perfectly hybridized to a complementary nucleotide; and
d) determining if any reaction occurred, wherein detecting a reaction of phosphorylated 5′ terminal nucleotide perfectly hybridized to a complementary nucleotide indicates the discrimination probe hybridized perfectly to the target nucleic acid molecule at the 5′ terminal nucleotide and that the allele present in the target nucleic acid molecule is the allele that has the single nucleotide polymorphism complementary to the 5′ terminus of the discrimination probe.

7. The assay of claim 1 comprising a reaction specific for unblocked 3′ terminal nucleotide perfectly hybridized to a complementary nucleotide or a reaction being specific for phosphorylated 5′ terminal nucleotide perfectly hybridized to a complementary nucleotide, wherein the reaction specific for unblocked 3′ terminal nucleotide perfectly hybridized to a complementary nucleotide or the reaction being specific for phosphorylated 5′ terminal nucleotide perfectly hybridized to a complementary nucleotide is an enzymatic ligation reaction of a labeling nucleotide or oligonucleotide to a discrimination probe or a discrimination extender having an unblocked 3′ terminal nucleotide perfectly hybridized to a complementary nucleotide or the reaction being specific for phosphorylated 5′ terminal nucleotide perfectly hybridized to a complementary nucleotide.

8. The assay of claim 1 comprising a reaction specific for unblocked 3′ terminal nucleotide perfectly hybridized to a complementary nucleotide or the reaction being specific for phosphorylated 5′ terminal nucleotide perfectly hybridized to a complementary nucleotide, wherein the reaction specific for unblocked 3′ terminal nucleotide perfectly hybridized to a complementary nucleotide or the reaction being specific for phosphorylated 5′ terminal nucleotide perfectly hybridized to a complementary nucleotide is a polymerase catalyzed primer extension reaction comprising incorporation of labeled nucleotides.

9. The assay of claim 1 comprising a reaction specific for unblocked 3′ terminal nucleotide perfectly hybridized to a complementary nucleotide, wherein the reaction specific for unblocked 3′ terminal nucleotide perfectly hybridized to a complementary nucleotide is a polymerase catalyzed primer extension reaction comprising incorporation of labeled nucleotides.

10. The assay of claim 1 comprising a reaction specific for unblocked 3′ terminal nucleotide perfectly hybridized to a complementary nucleotide, wherein the reaction specific for unblocked 3′ terminal nucleotide perfectly hybridized to a complementary nucleotide is a polymerase catalyzed single base extension reaction comprising incorporation of labeled nucleotides.

11. The assay of claim 1 comprising a reaction specific for unblocked 3′ terminal nucleotide perfectly hybridized to a complementary nucleotide or the reaction being specific for phosphorylated 5′ terminal nucleotide perfectly hybridized to a complementary nucleotide, wherein the reaction specific for unblocked 3′ terminal nucleotide perfectly hybridized to a complementary nucleotide or the reaction being specific for phosphorylated 5′ terminal nucleotide perfectly hybridized to a complementary nucleotide is a chemical ligation reaction of a labeling entity to a discrimination probe or a discrimination extender having an unblocked 3′ terminal nucleotide perfectly hybridized to a complementary nucleotide or the reaction being specific for phosphorylated 5′ terminal nucleotide perfectly hybridized to a complementary nucleotide.

12. The assay of claim 11 wherein the labeled entity is a labeled oligonucleotide.

13. The assay of any of claim 12 wherein labeled nucleotides are labeled with biotin, a fluorophore, a nanoparticle, or an enzymes.

14. The assay of any of claim 7 wherein labeled nucleotides are labeled with biotin, a fluorophore, a nanoparticle, or an enzymes.

15. The assay of claim 1 wherein the solid support is a bead, a planar surface, a metallic particle, or a gel matrix.

16. The assay of claim 1 wherein determination of whether a reaction occurred use optical, electrical, mechanical, or magnetic methods.

17. The assay of claim 1 wherein the assay is a multiplex assay to identify the presence of multiple genes having alleles that comprise a single nucleotide polymorphism, comprising an assay that comprises multiple discrimination probes that are each specific for a single gene that has alleles that comprise a single nucleotide polymorphism and discrimination probes and target capture probes are spatially separated on specific spots on a solid support, wherein the assay comprises multiple discrimination probes that are each specific for a single gene that has alleles that comprise a single nucleotide polymorphism and discrimination probes and target capture probes are spatially separated on specific spots on a solid support, or the assay comprises multiple discrimination extenders that are each specific for a single gene that has alleles that comprise a single nucleotide polymorphism and capture probes and target capture probes are spatially separated on specific spots on a solid support that are immobilized to the solid support in a spatial separated n or discrimination extenders.

18. The assay of claim 1 wherein a discrimination extender is hybridized to a capture probe immobilized to the solid support prior to contacting with the target nucleic acid molecule.

19. The assay of claim 18 wherein the target capture extenders are mixed with the target nucleic acid molecule prior to contacting with the target capture probes immobilized on the solid support.

20. The assay of claim 18 wherein the target capture extenders are mixed with the target capture probes immobilized on the solid support prior to contacting with the target nucleic acid molecule.

21. A kit comprising a solid support selected from the group consisting of a solid support that comprises:
a) a capture probe and one or more target capture probes linked to the solid support at the 3′ terminus directly or with spacers, one or more target capture extenders with blocked 3′ termini and sequences complementary to a sequence on a target capture probe and a target nucleic acid molecule, and a discrimination extender with an unblocked 3′ terminus that and a sequence complementary to a sequence on a capture probe and a target nucleic acid molecule wherein the nucleotide at the 3′ terminus of the discrimination extender is complementary to a single nucleotide polymorphism position of an allele;
b) a capture probe and one or more target capture probes linked to the solid support at the 5′ terminus directly or with spacers, one or more target capture extenders with unphosphorylated 5′ termini and sequences complementary to a sequence on a target capture probe and a target nucleic acid molecule, and a discrimination extender with an phosphorylated 5′ terminus and a sequence complementary to a sequence on a capture probe and a target nucleic acid molecule wherein the nucleotide at the 5′ terminus of discrimination extender is complementary to a single nucleotide polymorphism position of an allele;
c) a capture probe linked to the solid support at the 3′ termini directly or with spacers, one or more target capture probes with blocked 3′ termini and linked to the solid support at the 5′ terminus directly or with spacers, one or more target capture extenders with blocked 3′ termini and sequences complementary to a sequence on a target capture probe and a target nucleic acid molecule, and a discrimination extender with an unblocked 3′ terminus and a sequence complementary to a sequence on a capture probe and a target nucleic acid molecule wherein the nucleotide at the 3′ terminus of the discrimination extender is complementary to a single nucleotide polymorphism position of an allele; and combination thereof.

22. A kit of claim 21 wherein the solid support comprises more than one different capture probe that hybridizes to different discrimination extenders, each different capture probe spatially separated at identifiable locations and different discrimination extenders having termini complementary to a single nucleotide polymorphism position of an allele of different genes.

23. A solid support selected from the group consisting of:
a) a solid support comprising a discrimination probe linked to the solid support at the 5′ termini directly or with spacers, one or more target capture probes with blocked 3′ termini and linked to the solid support at the 5′ terminus directly or with spacers, wherein a sequence of a target probe is complementary to a sequence on a target nucleic acid molecule and a sequence on the discrimination probe is complementary to a sequence on the target nucleic acid molecule wherein the nucleotide at the 3′ terminus of the discrimination probe is unblocked and complementary to a single nucleotide polymorphism position of an allele;
b) a solid support comprising a discrimination probe linked to the solid support at the 3′ termini directly or with spacers, one or more target capture probes with unphosphorylated 5′ termini and linked to the solid support at the 3′ terminus directly or with spacers, wherein a sequence of a target probe is complementary to a sequence on a target nucleic acid molecule and a sequence on the discrimination probe is complementary to a sequence on the target nucleic acid molecule wherein the nucleotide at the 5′ terminus of the discrimination probe is phosphorylated and complementary to a single nucleotide polymorphism position of an allele; and combinations thereof.

24. A solid support of claim 23 wherein the solid support comprises more than one different discrimination probe, each different discrimination probe spatially separated at identifiable locations and different discrimination extenders having termini complementary to a single nucleotide polymorphism position of an allele of different genes.
